(19) United States
(12) Patent Application Publication
(10) Pub. No.: US 2024/0151737 A1
(43) Pub. Date: May 9, 2024

(54) CONFIGURABLE DIAGNOSTIC PLATFORM SYSTEMS AND METHODS FOR PERFORMING CHEMICAL TEST ASSAYS

(71) Applicant: PEEK TECHNOLOGIES, INC., Santa Clara, CA (US)

(72) Inventors: Wilma MANGAN, Santa Clara, CA (US); Alton B. OTIS, Jr., Santa Clara, CA (US); Tai NGO, Santa Clara, CA (US)

(73) Assignee: PEEK TECHNOLOGIES, INC., Santa Clara, CA (US)

(21) Appl. No.: 18/546,756

(22) PCT Filed: Feb. 17, 2022
(86) PCT No.: PCT/US2022/016693

§ 371 Date: Aug. 16, 2023

Related U.S. Application Data
(60) Provisional application No. 63/150,994, filed on Feb. 18, 2021.

(51) Int. Cl. G01N 35/00

(52) U.S. Cl. CPC G01N 35/00732; G01N 2035/00752; G01N 2035/00851; G01N 2035/0091

(57) ABSTRACT

Provided are methods and systems that relate to configuring a diagnostic analyzer. The diagnostic analyzer may receive an identifier from a dual design test cartridge. The parameter module disposed within the diagnostic analyzer may determine parameters corresponding to the received test cartridge identifier.

### CROSS-REFERENCE

This application claims the benefit of U.S. Provisional Application No. 63/150,994, filed on Feb. 18, 2021, which is entirely incorporated herein by reference.

### BACKGROUND

In the medical or alcoholic beverage-making industries, there is a need to have different diagnoses and/or tests performed regularly with a high level of accuracy.

### SUMMARY

The present subject matter relates to multiplex diagnostic platform system and more particularly to a configurable system for performing different chemical test assays (e.g., single use chemical test assays) on one portable instrument (e.g., an analyzer).

Qualitative and quantitative immuno- and chemical assays may be used as important tools in the medical and industrial industries. These methods may be used for the diagnosis of disease conditions, detection of analytes, and for the detection of microbes, such as bacteria.

Conventionally, the diagnostic assays are performed in laboratory settings, and involve the use of sophisticated and expensive equipment, that may require specially-trained personnel for their operation. Further, the assay results may sometimes be unavailable for days or weeks after the samples have been obtained. The conventionally-available diagnostic assays are thus costly, time consuming, and not convenient.

Attempts have been made to develop less costly assays. For example, a typical home self-test for detecting blood components requires the patient to prick a finger with a sterilized lancet, apply a drop of blood sample to a sample application area on the test cartridge, and then wait for the results. Assays that use other fluids, such as water samples essentially work in a similar manner. Conventionally, different instruments (e.g., test cartridges, analyzers, etc) are needed for performing different tests, which increases the costs of performing multiple tests. Additionally, a number of testing/diagnostic devices claim to be designed for lay person are still require a special amount of training/skill to prepare the test samples and interpret the test results. Some other devices are designed such that a typical layperson can perform the assays correctly with very little training. However, these assay systems generally suffer from low accuracy or require a number of preparative steps may be performed that could compromise the test results and are thus not convenient.

One of the preparative steps involves configuring the assay system (e.g., an analyzer) for a particular test or a particular type of test strip, so that one analyzer may analyze and interpret multiple tests. If the assay is not configured properly, the results may be inaccurate if not misleading. Manual configuration may be prone to human error and adds another hurdle that the patient or end user has to pass before the sample may be tested.

In some instances, the present subject matter provides systems and methods for configuring an analyzer that performs diagnostic tests using a (e.g., single use) test cartridge (e.g., comprising at least one single use component and at least one recyclable component). In general, the test cartridge comprises one or more chemical test sites and a parameter module that stores test identifiers containing information about the test assay to be performed at each site. Each test identifier is used to configure the analyzer to perform the associated diagnostic test and contains all of the calibration and result computation data for the test. The tests performed at the test sites are called the primary tests. In addition to the primary test identifiers the parameter module may contain secondary test identifiers. Secondary tests use the results from the primary tests to compute secondary test values that further expand the usefulness of the cartridge. In addition to the test identifiers the parameter module may also store the cartridge name, serial number, test lot numbers, expiration date, test temperature and other information (e.g., test configuration, calibration coefficient, etc.) that can be used to track the production of the cartridge and perform the tests.

The analyzer comprises a diagnostic controller, a parameter module, a storage module, a communications module, a heater module, and an optical processing module. The diagnostic controller reads information from the test cartridge's parameter module and directs one or more test firmware modules in the analyzer to execute test functions at the cartridge's test sites using the primary test identifiers. After the primary test results are computed any secondary test results specified in the parameter module are computed. When all the computations are complete the test results are saved in the storage module and displayed for the operator. The heater module is used to warm the test sites as required by the primary tests as specified in the parameter module. The optical processing module illuminates the test sites and reads reflected light to determine the degree of color change. This information is passed to the test firmware modules to monitor the test progress and determine when the test is complete, and the results are to be computed. The communication module provides the means for transmitting test results to a local smartphone or tablet via Bluetooth. cellular signal, or to a remote server via Wi-Fi. The operator can select these options while viewing the current test results or at a later time by reviewing test results stored in the storage module.

Provided herein is a configurable diagnostic test system comprising a test cartridge, wherein the test cartridge comprises a configurable parameter module, and an analyzer comprising a diagnostic controller, the diagnostic controller communicatively coupled with the configurable parameter module of the test cartridge, wherein the diagnostic controller (i) determines a configuration parameter for the test cartridge based on an identifier received from the configurable parameter module, and (ii) configures the analyzer to perform a test based on the determined configuration parameter, wherein the configuration parameter comprises a predefined temperature for the test. In some embodiments, the analyzer further comprises a test firmware module. In some embodiments, the test firmware module provides configuration for the analyzer based on the received identifier. In some embodiments, the test firmware module prevents use of the cartridge based upon information in the received identifier. In some embodiments, the test firmware module provides configuration for the analyzer based on the determined configuration parameter. In some embodiments, the identifier comprises a first test identifier. In some embodiments, the diagnostic controller configures the analyzer to perform a first test at one of the plurality of test sites based on the first test identifier. In some embodiments, the diagnostic controller configures the analyzer to perform a second test based on the test result from the first test. In some embodiments, the analyzer further comprises an optical processing module. In some embodiments, the optical processing module determines an insertion of the test cartridge. In some embodiments, the optical processing module determines the degree of color change by illuminating one or more of a plurality of test sites of the test cartridge. In some embodiments, the configurable diagnostic test system further comprises a heater module. In some embodiments, the heater module warms one or more of test sites based on a third identifier received from the configurable parameter module. In some embodiments, the test cartridge is portable and can be placed close or inside of the analyzer. In some embodiments, the analyzer comprises a test cartridge containment to receive the insertion of the test cartridge. In some embodiments, the test cartridge comprises a plurality of ports for receiving test samples. In some embodiments, the test cartridge comprises a plurality of apertures, and a test result may be viewed through the plurality of apertures. In some embodiments, the analyzer comprises a display screen, the display screen displays a test result based on a predetermined set of the rules associated with a test. In some embodiments, the diagnostic controller receives the identifier from the configurable parameter module through a RFID tag reader. In some embodiments, the configurable parameter module comprises a barcode, and the diagnostic controller receives the identifier from the configurable parameter module by scanning the barcode.

Provided herein is a configurable diagnostic test method, comprising: (a) inserting a test cartridge into a cartridge containment of an analyzer; (b) receiving a set of parameters from the test cartridge; (c) configuring the analyzer based on the received set of parameters; (d) receiving a test sample at the test cartridge when the test cartridge is at a predefined temperature, and (d) outputting the test result produced by the analyzer. In some embodiments, the configurable diagnostic test method further comprises receiving an identifier (e.g., such as to label the cartridge as having been used) (e.g., sent by the analyzer and received by the cartridge, such as a writable RFID present within the cartridge). In specific embodiments, once received, the cartridge is rendered unusable (e.g., based on information in the identifier). In some embodiments, the method further comprises preventing use of the cartridge based upon information in the identifier. In some embodiments, the configurable diagnostic test method further comprises heating the test cartridge by a heater module of the analyzer to the predefined temperature. In some embodiments, the configurable diagnostic test method further comprises processing a second test result based on the output test result.

Provided herein is a configurable diagnostic test method, comprising (a) receiving, at an analyzer, an identifier from a test cartridge; (b) determining a configuration for the analyzer based on the received identifier; (c) inquiring a firmware module of the analyzer for a set of rules associated with the received identifier; (d) configuring the analyzer based on the set of rules; (e) receiving a test sample to perform a test, and (f) providing instructions to a user based on the set of rules. In some embodiments, the configurable diagnostic test method further comprises providing instruction to the user based on the set of rules when a predefined amount of time passed since received the test sample.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a non-limiting example of a base system and a test cartridge utilized by embodiments described herein;

FIG. 2 illustrates a non-limiting example of the test cartridge utilized by embodiments described herein;

FIG. 3 illustrates a non-limiting example of the test sites of the test cartridge utilized by embodiments described herein;

FIG. 4 illustrates a non-limiting example of the reaction wells of the test cartridge utilized by embodiments described herein.

FIG. 5 illustrates a non-limiting example of a schematic diagram; in this case, a schematic diagram illustrating hardware aspects of the platforms, systems, and methods for providing a diagnostic and/or testing platform according to embodiments described herein;

FIG. 6 illustrates a non-limiting example of a schematic diagram; in this case, a schematic diagram illustrating architecture and procedural aspects of the platforms, systems, and methods for providing a diagnostic and/or testing platform according to embodiments described herein; and

FIGS. 7A and 7B show non-limiting examples of a process flow diagram; in this case, a process flow diagram illustrating testing process according to embodiments described herein.

FIG. 8 shows a non-limiting example of a process flow diagram; in this case, a process flow diagram illustrating diagnostic testing process according to embodiments described herein.

FIG. 9 shows non-limiting example of a process flow diagram; in this case, a process flow diagram illustrating diagnostic testing process according to embodiments described herein.

FIG. 10 illustrates example computer architecture applicable to any computer system discussed herein.

### DETAILED DESCRIPTION

### Certain Definitions

Unless otherwise defined, all technical terms used herein have the same meaning as commonly understood by one of ordinary skill in the art to which the present subject matter belongs.

The terms “specifically binds to” or “specifically immunoreactive with” refers to a binding reaction which is determinative of the presence of the target analyte in the presence of a heterogeneous population of proteins and other biologics. Thus, under designated assay conditions, the specified binding moieties bind preferentially to a particular target analyte and do not bind in a significant amount to other components present in a test sample. Specific binding to a target analyte under such conditions may require a binding moiety that is selected for its specificity for a particular target analyte.

As used herein, the terms “label” and “detectable label” refer to a molecule capable of detection, including, but not limited to, radioactive isotopes, fluoresces, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens) and the like.

“Specific” in reference to the binding of two molecules or a molecule and a complex of molecules refers to the specific recognition of one for the other and the formation of a stable complex as compared to substantially less recognition of other molecules and the lack of formation of stable complexes with such other molecules. Exemplary of specific binding are antibody-antigen interactions, enzyme-substrate interactions, polynucleotide hybridizations and/or formation of duplexes, cellular receptor-ligand interactions, and so forth.

The figures (FIG.) and the following description relate to some embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

As used herein any reference to “one embodiment,” “an embodiment,” or “some embodiments” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. It should be understood that these terms are not intended as synonyms for each other. For example, some embodiments may be described using the term “connected” to indicate that two or more elements are in direct physical or electrical contact with each other. In another example, some embodiments may be described using the term “coupled” to indicate that two or more elements are in direct physical or electrical contact. The term “coupled,” however, may also mean that two or more elements are not in direct physical or electrical contact with each other, but yet still co-operate or interact with each other. The embodiments are not limited in this context.

Also, some embodiments of the invention may be further divided into logical modules. One of ordinary skill in the art will understand that these modules can be implemented in hardware, firmware and/or software. In one embodiment, the modules are implemented in form of computer instructions stored in a computer readable medium when executed by a processor cause the processor to implement the functionality of the module. Additionally, one of ordinary skill in the art will recognize that a computer or another machine with instructions to implement the functionality of one or more logical modules is not a general-purpose computer. Instead, the machine is adapted to implement the functionality of a particular module.

As used herein, the terms “comprises,” “comprising,” “includes,” “including,” “has,” “having” or any other variation thereof, are intended to cover a non-exclusive inclusion. For example, a process, method, article or apparatus that comprises a list of elements is not necessarily limited to only those elements but may include other elements not expressly listed or inherent to such process, method, article or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

### Overview

The invention pertains to a single use test cartridge that can be used for performing qualitative and quantitative immuno- and chemical assays. On the test cartridge are at least three wells, where the wells can be in fluidic communication with each other via capillary channels. In one well is placed the sample, preferably a liquid sample, for analysis. The sample fluid moves into the other two wells (also referred to herein as an active site) via the capillary channels. One of the wells can serve as a standard that measures the total analyte in the sample. The other well can serve as the reaction well, where individual components of the sample can be identified.

The single use test cartridge is inserted into an analyzer (also referred to herein as a “base”) and the analyzer retrieves the necessary information from the test cartridge. The analyzer then configures itself to perform a diagnostic test using that particular single use single use test cartridge.

The single use test cartridge can be placed in an analyzer that detects the individual components of the sample and the total analytes in the sample. The analyzer includes a display system that can display the results of the analysis as well as provide instructions during the operation of the assay.

In one application, the percent total reduced sugar such as glucose and fructose in grape juice can be determined. Grape juice can be deposited in the sample well. The present subject matter includes testing platforms, devices and systems that may test grape juice, must and alcohol during the testing process, or at the conclusion of fermentation. The juice is moved through a stack of pretreated membranes laminated in a test cartridge. In one area of the test cartridge can be impregnated with reagents that enzymatically measures glucose and fructose. In another area of the test cartridge can be impregnated with reagents that enzymatically measures glucose. When the juice moves into these enzymatic areas a detectable label generates a visual color change proportional to the quantity of analyte present that can be used to calculate the concentration of glucose and fructose. Fructose is measured by subtracting color generated from the reaction zone that measures the sum of glucose and fructose with the reaction zone that measures only glucose. The results for both glucose and fructose can be displayed on the display system.

### System Overview

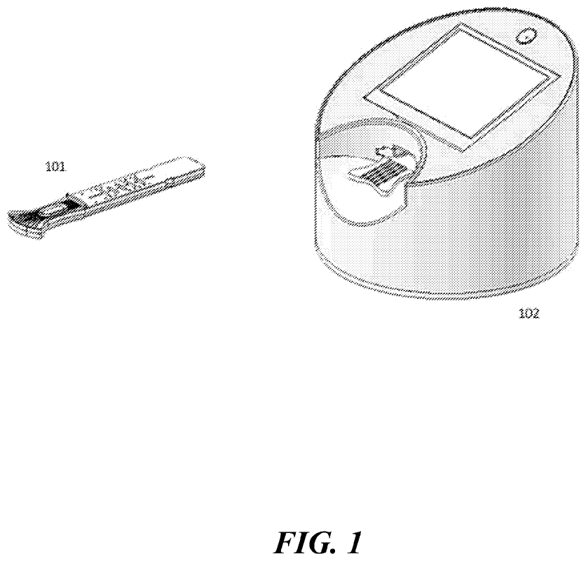

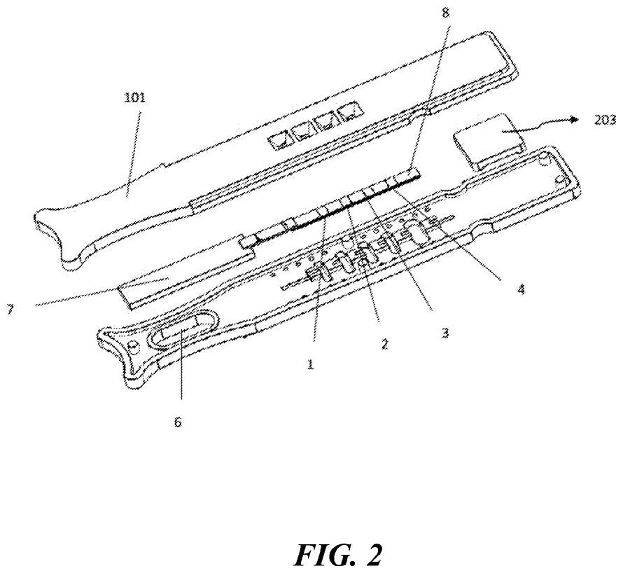

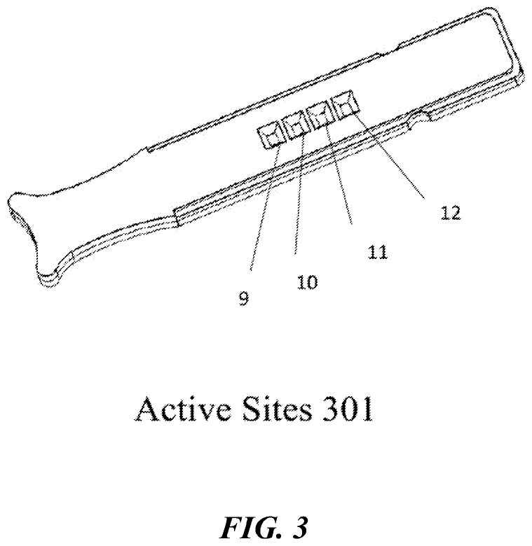

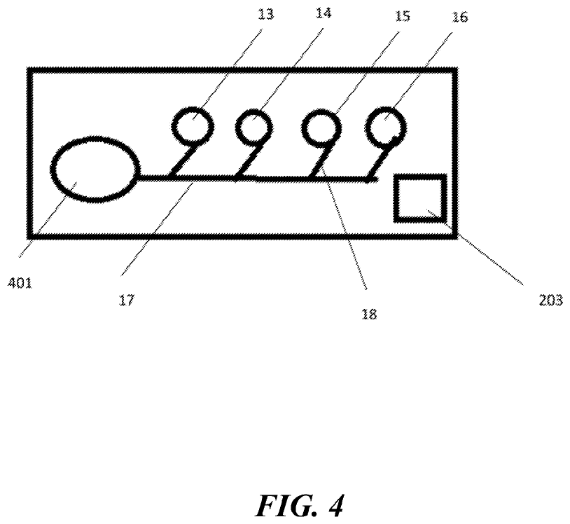

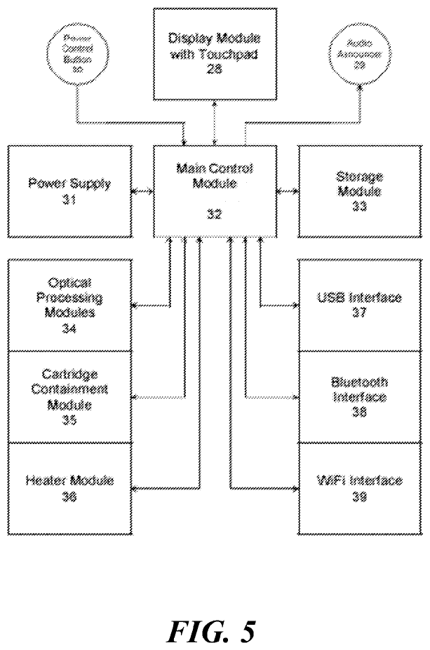

Referring to FIG. 1, a diagram is shown depicting configurable diagnostic test system. The present subject matter provides a test cartridge 101 (e.g., a single use cartridge), an analyzer 102 (e.g., a base) and a configurable diagnostic test system comprising a single use test cartridge 101 and an analyzer 102. The single use test cartridge 101 is illustrated in more detail below in FIG. 2, FIG. 3 and FIG. 4; the base is described in FIG. 5. As shown in FIG. 1, the test cartridge 101 may be a standalone component, which is not part of the analyzer. In some embodiments, the analyzer 102 may have an opening or a containment in which the test cartridge 101 may be placed. Once the test cartridge 101 is inserted into the opening or containment of the analyzer 102, the analyzer 102 may configure itself to perform or aid the test cartridge 101 to perform certain tests. In some other embodiments, the test cartridge 101 may not need to be placed in the analyzer 102 to perform a scheduled test. For example, the analyzer 102 may receive the test results from the test cartridge 101 wirelessly. Whether or not a test cartridge 101 needs to be placed inside of the analyzer 102 to perform the particular test that the test cartridge is configured to perform may be based on the type of tests the test cartridge 101 is to perform, or it may be based on the model of the analyzer 102.

Referring to FIG. 2, a diagram is shown depicting an exemplary test cartridge 101. The test cartridge 101 includes a test cartridge parameter module 203 (e.g., a configurable parameter module) that stores information associated the with test cartridge 101. This test cartridge parameter module 203 can be any module capable of storing information for later retrieval by a reader (e.g., a reader in the analyzer 102, an external reader, a reader contained in the test cartridge 101, etc.). For example, the test cartridge parameter module 203 can be an integrated circuit capable of storing and transmitting information through a wired or wireless connection, an RFID tag, or a barcode etc. In one embodiment, the test cartridge parameter module 203 specifies the test(s) contained in the cartridge 101 and the configuration parameters for these test(s) to be performed by the analyzer 102. The parameters and the tests are described further below. In one embodiment, the analyzer 102 may include an RFID tag reader, and when the test cartridge 101 is placed close to the analyzer 102, the analyzer 102 may read the information from the RFID tag of the test cartridge 101, and thus configure itself with that information. In some embodiments, the RFID tag reader is writeable. In another embodiment, the analyzer 102 may include a barcode reader, and when the test cartridge 101 is placed closed to the barcode reader of the analyzer 102, the analyzer 102 may read the information from the barcode of the test cartridge 101, and thus configure itself with that information.

Regardless of how the information is retrieved, the analyzer 102 configures itself using the retrieved information with minimal input from the user. In some embodiments, the analyzer 102 may configure itself without any user intervention or input. Accordingly, the configuration of analyzer 102 becomes an effortless process that increases test accuracy and reduces the possibility of test errors.

One aspect of the single use test cartridge 101 is illustrated in FIG. 2. The single use test cartridge 101 can be made by joining together two or more solid supports with grooves present in at least one of the supports. The solid support can be rectangular, circular, oval, or any shape. The support can be made from a suitable material that is selected on its properties, such as good thermal conductivity, clarity for optical transmission, mechanical properties for easy welding, surface properties that allow for uniform coating and stability of reagent, and neutrality to the liquid medium to prevent interference with the assay. For this purpose, suitable plastics include those with high free surface energies and low water absorption, including PETG, polyester (Mylar®), polycarbonate (Lexan®), polyvinyl chloride, polystyrene, SAN, acrylonitrile-butadiene-styrene (ABS), particularly ABS supplied by Borg Warner under the trade name Cyclolac, among others. When the solid support is a hydrophobic plastic, it can be treated by art-known methods to render the surfaces hydrophilic, such as by plasma etching and by corona treatment. Alternatively, or equivalently, a commercially available molded solid support can be used in the practice of the invention.

An assembled single use test cartridge is illustrated in FIGS. 2 and 3, wherein an absorbent pad 7 is placed in between the two (or more) solid supports. Test membrane impregnated with immune-chemistries, enzymes or chemicals 8 is placed on one or both ends of the absorbent pad 7. capture zones 1, 2, 3, and 4 are along the test membrane 8. A sample application port 6 is on one or both of the solid supports.

Referring to FIG. 3, a diagram is shown depicting an exemplary portion of the test cartridge 101, wherein the capture zones 9, 10, 11, and 12 are on the test membrane 8. These captures zones collectively formed active sites 301. As apparent from FIGS. 1-3, the test cartridge parameter module 203 can be placed at various different places on the test cartridge 101 and the placement of test cartridge parameter module 203 is not meant to be limited by the illustrated placement in FIGS. 1-3.

Referring to FIG. 4, a diagram is shown depicting an exemplary portion of the test cartridge 101, wherein the test cartridge 101 is formed by joining two solid supports (as shown in FIG. 2). The test cartridge parameter module 203 can reside on either of the two solid supports or in between. Moreover, at least one of the solid supports has grooves or cavities that serve as the reaction chambers 13, 14, 15, and 16 and capillary channels 17 and 18. The grooves can be any geometric shape and are preferably circular. The grooves dimensions have sufficient volume to hold the samples and to allow for the reaction to occur. Thus, if circular grooves are used they can have any suitable diameter, such as a diameter of greater or equal to 0.01 mm, depending on the length and width of the support. The cross section of the grooves can be easily determined by one of skill in the art. In one aspect of the present subject matter, one of the support pieces (e.g., the solid supports) has holes drilled through to the grooves where the holes serve as the vent holes. Further, the holes can allow access to the well where the sample will be placed, such as the sample application well 401.

The test parameters from the test cartridge parameter module 203 contains test information such as lot numbers, expiration dates, test types, test the calibration parameters and computing coefficients for each active site 301 (e.g., capture zones 9, 10, 11, and 12 in FIG. 3). In one embodiment, the cartridge parameter module 203 also contains associative secondary computing parameters between active sites. In another embodiment the cartridge parameter module 203 also contains test specific codes that configure the analyzer 102 (e.g., base).

For embodiments in which the cartridge parameter module 203 is implemented with a writable device, such as an RFID tag, the analyzer may store a code in the cartridge parameter module 203 to indicate cartridge 101 has been previously used. Upon reading the code the analyzer would signal the user that the cartridge has been used and prevent its further use.

Referring to FIG. 5, a block diagram is shown depicting exemplary hardware modules of an exemplary analyzer 102. The hardware modules include a display module with integrated touch pad 28, an audio announcer 29, a power control button 30, a power supply 31, main control module (a microprocessor with embedded program memory) 32, a storage module 33, a set of two or more optical processing modules 34, a cartridge containment module 35, a heater module 36, a USB interface 37, a Bluetooth interface 38 and a Wi-Fi interface 39.

The display module 28 contains a graphic liquid crystal display with an integrated touch pad data entry overlay. The display could be any type that provides graphic images or even a text only display. The touch pad could be a separate mechanical or electronic keyboard. The audio announcer 29 is a loudspeaker but could also be a beeper. The purpose of the announcer 29 is to notify the user of certain events that may require attention. Storage module 33 can, among other functions, save test results for later review by a user.

The power control button 30 is a mechanical button but could also be a capacitively sensed button. Power supply 31 takes power from internal batteries or an external power source and converts it into the regulated voltages required by the other modules in the analyzer. The batteries may be primary single use types or rechargeable types. The USB interface 37, Bluetooth interface 38 and Wi-Fi interface 39 provide means to transmit data (e.g., test results, time/date of the test or test result, location, lot information, etc.) to external computers, smart phones, tablets or data servers. The USB interface may also be used to upgrade the firmware in main control module 32.

The cartridge containment module 35 may hold the test cartridge 101 prior to and during testing. The containment module 35 may include a sensor that detects the full insertion of a cartridge. In one embodiment, this is a mechanical switch however other types of sensors may be used. The containment module 35 may also include a device that reads the information of the parameter module 203 of the test cartridge 101. In one embodiment, the device may be an RFID tag reader. In another embodiment, the device may be a barcode reader. In some other embodiments, the device may be a combination of a variety of readers, such as RFID reader, barcode reader. In other embodiments, the device may be a Bluetooth receiver that may receive information from the parameter module 203 of the test cartridge 101. The information retrieved from the parameter module 203 of the test cartridge 101 may be transmitted to main control module 32, and the main control module 32 may use the retrieved information to configure the analyzer 102.

The optical processing modules 34 illuminates the capture zones and measures the amount of reflected light from each of the test sites on test cartridge 101. The detection mechanism is not limited to a reflected light method and other methods such as fluorescent emissions from UV stimulation or electrical sensing could also be employed.

The heater module 36 is used to heat the test sites in test cartridge 101 to a temperature specified in the contents of parameter module 203 in the test cartridge. In one embodiment, the heater module 36 is a ceramic plate with a resistive heating element applied to one side and a thermistor on the same side to provide temperature feedback to main control module 32. The other side of the ceramic plate is placed in intimate contact with the test cartridge to provide the heating function. Other heating methods such as infrared light could also be employed.

The optical processing modules 34 and heater module 36 may be placed on either side of containment module 35. In the preferred embodiment the heater module is above the cartridge and the optical processing modules are below. It is also possible to place both modules on the same side of the single use cartridge.

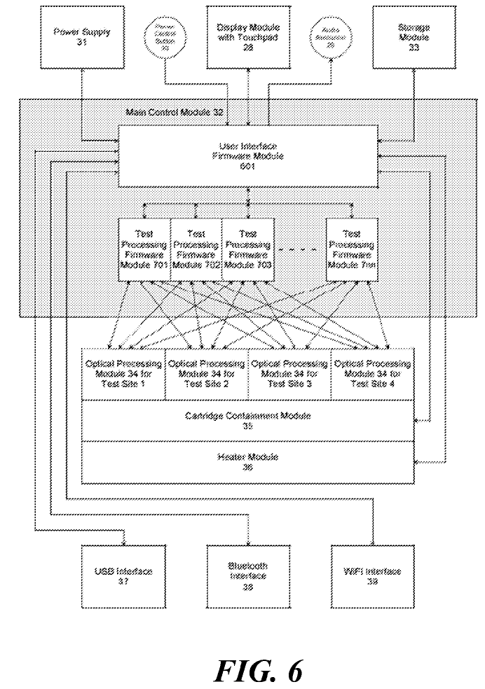

Referring to FIG. 6, a block diagram is shown depicting an exemplary firmware module of the analyzer 102. In one embodiment, the firmware module runs in the main control module 32 of the analyzer 102. The user interface (UI) firmware module 601 formats the content of the information shown on display module 28 and captures user key presses. Power control button 30 enables power to the main control module 32 which in turn enables power to the other components as needed. While power is enabled to main control module 32 pressing power control button 30 causes main control module 32 to disable power to all components then to itself; display module 28 turns off and the unit enters a very low power sleep mode.

User interface firmware module 601 may also control USB interface 37, Bluetooth interface 38, Wi-Fi interface 39, heater module 36 and cartridge containment module 35. Test processing firmware modules 701-7nn may control the optical processing modules 34, based on the firmware it contains, or as directed by the user interface module 601. Test processing firmware modules 701-7nn each perform a specific test type by controlling and reading data from optical processing module 34. These modules are selectively activated by UI firmware module 601 according to the test types specified in parameter module 203 of test cartridge 101. In one embodiment, one test processing module is assigned to each active site. Additional test processing firmware modules may be added by firmware upgrades.

### Operation Process: Example Test for Glucose and Fructose in Wine

The D-glucose and D-fructose content, often referred to as total reducing sugars, is considered a key quality parameter ahead of & during fermentation as it reflects the amount of sugar available to yeast for conversion into ethanol. By testing glucose and fructose in juice, winemakers can estimate the potential alcohol concentration of the finished wine. The total concentrations of glucose and fructose in harvested wine grapes is typically 17-26 grams per 100 milliliters.

A sample of pressed wine grapes or juice can be deposited in the sample application well 401 of the single use test cartridge 101 in FIG. 4. The juice moves to the active sites via capillary action. Active sites 1 and 3 (e.g., shown in FIG. 2) can be treated with chemicals specific to fructose and glucose or both glucose and fructose which generates a detectable color change proportional to the concentration of these analytes. Site 2 and 4 (e.g., shown in FIG. 2) be treated without the chemicals specific to glucose and fructose and be used as a blank. Chemical specific to glucose or fructose can be enzymatic base or general chemistry base.

In one example, an enzymatically based reaction for glucose and fructose is described below:

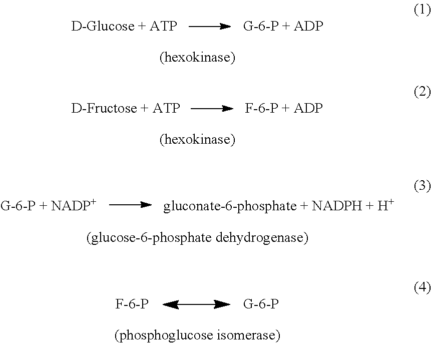

In this enzymatic reaction, chemistries steps 1-3 are impregnated onto active site 1 of the test cartridge 101 and steps 1-4 are impregnated onto active site 3. In addition, a detectable label such as NBT (Nitrotetrazolium blue chloride) and PMS (phenazine methosulphate) are added to each active to generate a color at approximate 560 nm. The color change measurements at a predetermined time, can be correlated to the concentration of either glucose or fructose as described below.

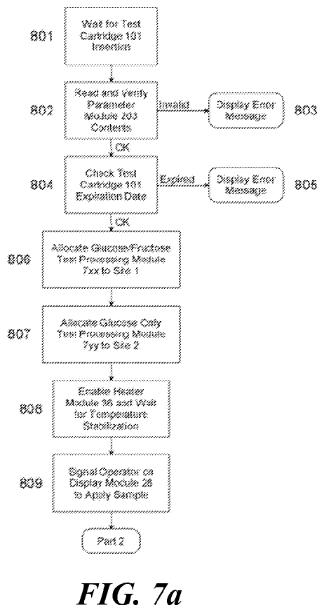

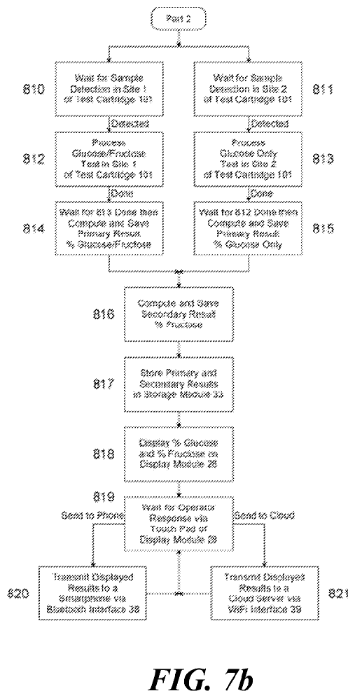

Referring to FIG. 7a and FIG. 7b, in a particular embodiment, a process for detecting Glucose and Fructose in grape juice begins with operation 801: waiting for the operator to insert a single use test cartridge 101 into the cartridge containment module 35. Once the cartridge insertion is detected the process continues to operation 802 where the contents of the parameter module 203 is read and verified. If the contents are invalid the process ends with an error at 803 and displays and error message on display module 28. Next the process checks the expiration date of test cartridge 101 at operation 804. If the cartridge has expired the process ends with an error at operation 805 and displays and error message on display module 28.

Next the contents read from cartridge parameter module 203 are checked to see if a used cartridge code is present. If it is the process ends with an error at operation 805 and displays and error message on display module 28. Otherwise if cartridge parameter module 203 is implemented with a writable device, a used cartridge code is written to the module to prevent reuse of the cartridge after the current test is complete.

From information stored in parameter module 203 the process assigns the Glucose/Fructose test processing module 7xx to site 1 at operation 806 and the Glucose only test processing module 7yy to site 2 at operation 807 where sites 1 and 2 are the only active sites in test cartridge 101. Next the process enables heater module 36 at operation 808 and waits until the heater temperature has stabilized at the value specified in parameter module 203 of test cartridge 101. Then at operation 809 the process signals the operator via display module 28 and/or audio announcement 29 to apply the sample to the sample entry well 401 of the test cartridge.

At operations 810 and 811 the two test processing modules independently wait until the sample is detected in their assigned site. The detection is made by noting when the reflected light in the site reaction chamber, 13 or 14, begins to darken indicating the presence of the sample. The detection at site 13 will occur before that at site 14 due to its proximity to the sample entry well 401. At operations 812 and 813 the two test processes analyze the reflected light at their respective sites to measure a change or rate of change in the reflected lights. The duration of operations 812 and 813 are dependent on the process complexity and the quantity of analyte in the sample and thus are asynchronous. To resynchronize the two processes, at 814 the process waits for the completion of operations 813 and at 815 the process waits for the completion of operation 812. In some instances, alternate sequences may be utilized, such as by transposing 814 and 815. At the point the independent test processing modules 7xx and 7yy are resynchronized.

At operation 814 the percentage of Glucose/Fructose is computed and at operation 815 the percentage of Glucose only is computed then at operation 816 secondary result percentage of Fructose is computed by subtracting the percentage of Glucose only from percentage of Glucose/Fructose. Next at operation 817 the primary and secondary results are stored in storage module 33 for later recall and review.

Finally at operation 818 the values of percentage of Glucose and percentage of Fructose are displayed on display module 28. At operation 819 the process waits for the operator to request transmission of the displayed results to either a smartphone or tablet via Bluetooth interface 38 or to cloud storage via Wi-Fi interface 39. Operations 820 and 821 perform these operations if so requested by the operator.

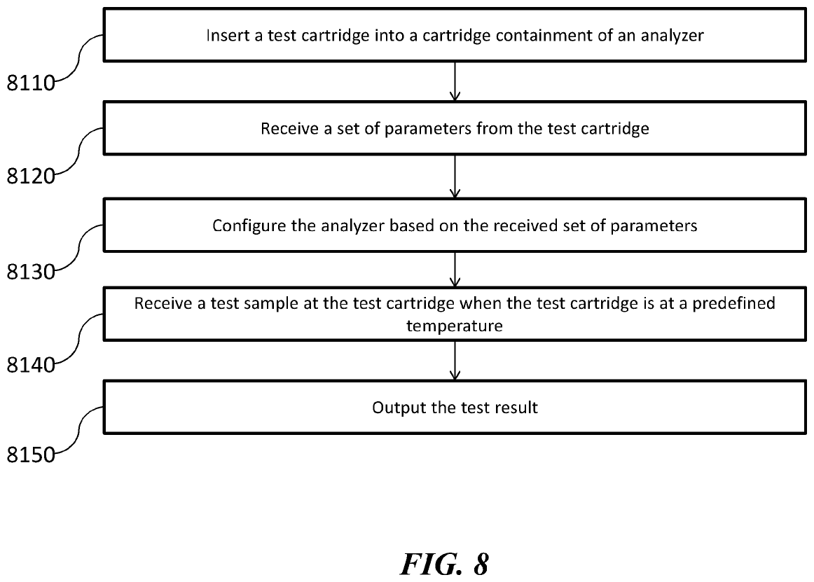

Referring to FIG. 8, in a particular embodiment, a process for illustrating diagnostic testing process begins with operation 8110 inserting a test cartridge into a cartridge containment of an analyzer. In this operation, the cartridge containment of the analyzer may include a sensor or a device for detecting the insertion of a test cartridge. The diagnostic testing process may proceed to receive a set of parameters from the test cartridge in operation 8120. In this operation, the analyzer may receive the set of parameters via a reader. For example, an RFID tag reader may be equipped in the analyzer so as to read the set of parameters stored in a RFID tag of the test cartridge. In other embodiments, other types of readers and/or transmission methods may be used to provide the same type of function. The diagnostic testing process may then proceed to configure the analyzer based on the received set of parameters in operation 8130. In this operation, the analyzer may retrieve rules from a firmware module based on the received set of parameters. Alternatively or additionally, the analyzer may configure itself based on the received set of parameters without inquiring any firmware or any other database. In the next operation 8140, the system receives a test sample at the test cartridge when the test cartridge is at a predefined temperature. In this operation, the analyzer may determine the temperature of the test cartridge is at a predefined temperature, and provide instructions or notifications via a display indicating a user may apply the test sample to the test cartridge now. In some embodiments, the process may further include an additional operation to heat/cool the test cartridge to the desired (e.g., predefined) temperature. A predefined temperature is a temperature that is suitable for a particular test that the test cartridge is about to perform. In some embodiments, this predefined temperature is one parameter of the set of parameters received from the test cartridge. As described above, in operation 8140, the test cartridge receives the test sample at the predefined temperature, and then the system may output the test result in operation 8150.

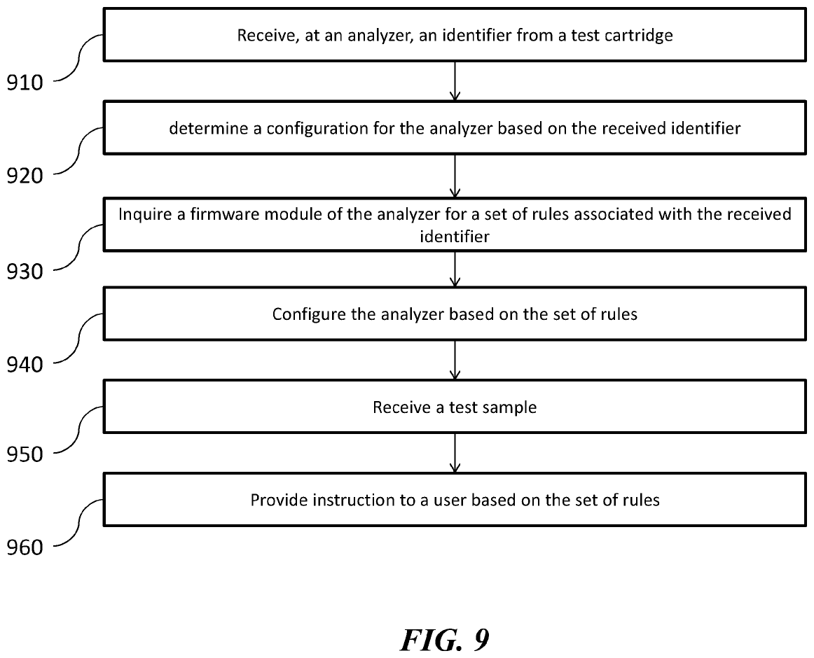

Referring to FIG. 9, in a particular embodiment, a process for illustrating diagnostic testing process begins with operation 910 by receiving an identifier from a test cartridge. The identifier may state what particular test this test cartridge is set to perform. Next, in operation 920, the system determines a configuration for the analyzer based on the received identifier. The diagnostic testing process proceeds to inquire 930 a firmware module of the analyzer for a set of rules associated with the received identifier. In some embodiments, there exist multiple firmware modules and one of the firmware modules may be chosen based on the received identifier. In some other embodiments, the firmware module stores a cluster of sets of rules associated with different identifiers respectively. In the next operation 940, the system configures the analyzer based on the set of the rules, wherein the set of rules may provide instructions to a user. In the next operation, the system receives 950 a test sample. As described above, the set of rules may include instructions to a user. For example, the system may instruct a user to apply test sample at a given time, when the test cartridge is at a given temperature, or when a given period of time has passed since the test cartridge received a first test sample, etc. The diagnostic testing process may provide these instructions to a user in operation 960. Thus, a user who is not a specially-trained laboratory worker may operation the diagnostic testing system in a consistent and accurate way and thus produce high-level accurate results.

### Computer Control Systems

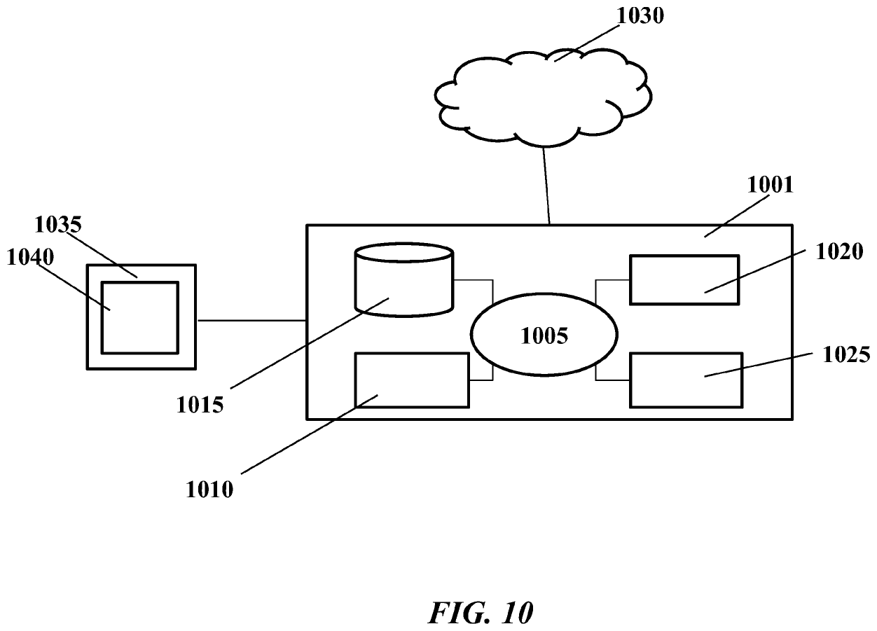

The present disclosure provides computer control systems that are programmed to implement methods of the disclosure. FIG. 10 shows a computer system 1001 that is programmed or otherwise configured to provide firmware function for the analyzer. The computer system 1001 can regulate various aspects of FIGS. 1-9 of the present disclosure, such as, for example, firmware module 601, flow chart illustrated in FIGS. 8-9, and various example applications of the diagnostic testing system described herein.

In some instances, the computer system 1001 may correspond to any node, such as a server, processor, or user device, described herein. The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020.

The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback. The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs, or any other data or information described herein. Files or information stored in the storage unit 1015 can include identifier index, test cartridge usage data and its operations. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., mobile device, smartphone, tablet). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can represent the dynamic segmentation platform 410 and include or be in communication with an electronic display 1035 that comprises a user interface (UI) 1040 for providing, for example, segment data, user data, content data, rule data, project related data, or any other data related to contents and project performance. In another embodiment, the computer system 1001 can represent one or more devices (e.g., an analyzer) that comprise a user interface 1040 for providing, for example, test results or instructions to the users. Examples of UI's include, without limitation, a graphical user interface (GUI) and web-based user interface.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A configurable diagnostic test system comprising:
a test cartridge, wherein the test cartridge comprises a configurable parameter module, and
an analyzer comprising a diagnostic controller, the diagnostic controller communicatively coupled with the configurable parameter module of the test cartridge, wherein the diagnostic controller (i) determines a configuration parameter for the test cartridge based on an identifier received from the configurable parameter module, and (ii) configures the analyzer to perform a test based on the determined configuration parameter, wherein the configuration parameter comprises a predefined temperature for the test.

2. The configurable diagnostic test system of claim 1, wherein the analyzer further comprises a test firmware module.

3. The configurable diagnostic test system of claim 2, wherein the test firmware module prevents use of the cartridge based upon information in the received identifier.

4. The configurable diagnostic test system of claim 2, wherein the test firmware module provides configuration for the analyzer based on the received identifier.

5. The configurable diagnostic test system of claim 2, wherein the test firmware module provides configuration for the analyzer based on the determined configuration parameter.

6. The configurable diagnostic test system of claim 2, wherein the identifier comprises a first test identifier.

7. The configurable diagnostic test system of claim 6, wherein the diagnostic controller configures the analyzer to perform a first test at one of the plurality of test sites based on the first test identifier.

8. The configurable diagnostic test system of claim 7, wherein the diagnostic controller configures the analyzer to perform a second test based on the test result from the first test.

9. The configurable diagnostic test system of claim 1, wherein the analyzer further comprises an optical processing module.

10. The configurable diagnostic test system of claim 9, wherein the optical processing module determines an insertion of the test cartridge.

11. The configurable diagnostic test system of claim 9, wherein the optical processing module determines the degree of color change by illuminating one or more of a plurality of test sites of the test cartridge.

12. The configurable diagnostic test system of claim 1, wherein the configurable diagnostic test system further comprises a heater module.

13. The configurable diagnostic test system of claim 12, wherein the heater module warms one or more of test sites based on a third identifier received from the configurable parameter module.

14. The configurable diagnostic test system of claim 1, wherein the test cartridge is portable and can be placed close or inside of the analyzer

15. The configurable diagnostic test system of claim 1, wherein the analyzer comprises a test cartridge containment to receive the insertion of the test cartridge.

16. The configurable diagnostic test system of claim 1, wherein the test cartridge comprises a plurality of ports for receiving test samples.

17. The configurable diagnostic test system of claim 1, wherein the test cartridge comprises a plurality of apertures, wherein a test result may be viewed through the plurality of apertures.

18. The configurable diagnostic test system of claim 1, the analyzer comprises a display screen, the display screen displays a test result based on a predetermined set of the rules associated with a test.

19. The configurable diagnostic test system of claim 1, the diagnostic controller receives the identifier from the configurable parameter module through a RFID tag reader.

20. The configurable diagnostic test system of claim 1, wherein the configurable parameter module comprises a barcode, and the diagnostic controller receives the identifier from the configurable parameter module by scanning the barcode.

21. A configurable diagnostic test method, comprising:
(a) inserting a test cartridge into a cartridge containment of an analyzer;
(b) receiving a set of parameters from the test cartridge;
(c) configuring the analyzer based on the received set of parameters;
(d) receiving a test sample at the test cartridge when the test cartridge is at a predefined temperature, and
(d) outputting the test result produced by the analyzer.

22. The configurable diagnostic test method of claim 21, further comprising receiving an identifier and preventing use of the cartridge based upon information in the identifier.

23. The configurable diagnostic test method of claim 21, further comprising heating the test cartridge by a heater module of the analyzer to the predefined temperature.

24. The configurable diagnostic test method of claim 21, further comprising processing a second test result based on the output test result.

25. A configurable diagnostic test method, comprising:
(a) receiving, at an analyzer, an identifier from a test cartridge;
(b) determining a configuration for the analyzer based on the received identifier;
(c) inquiring a firmware module of the analyzer for a set of rules associated with the received identifier;
(d) configuring the analyzer based on the set of rules;
(e) receiving a test sample to perform a test, and
(e) providing instructions to a user based on the set of rules.

26. The configurable diagnostic test method of claim 25, further comprising providing instruction to the user based on the set of rules when a predefined amount of time passed since received the test sample.
